(19) United States
(12) Patent Application Publication
(10) Pub. No.: US 2024/0279160 A1
(43) Pub. Date: Aug. 22, 2024

(54) METHOD FOR SYNTHESIZING 9-AMINOMETHYL TETRACYCLINE COMPOUNDS

(71) Applicant: HOVIONE SCIENTIA LIMITED, Co. Cork (IE)

(72) Inventors: Marina Ciriani, Lisboa (PT); Rudi Oliveira, Loures (PT); Rafael Antunes, Setúbal (PT); Carlos Afonso, Sobreda (PT)

(21) Appl. No.: 18/563,953

(22) PCT Filed: May 26, 2022
(86) PCT No.: PCT/GB2022/051341

§ 371 Date: Nov. 24, 2023

(30) Foreign Application Priority Data
May 26, 2021 (PT) 117254

(51) Int. Cl. C07C 237/26

(52) U.S. Cl. CPC C07C 237/26; C07C 2603/46

(57) ABSTRACT

A method for synthesizing 9-aminomethyl tetracycline compounds is disclosed. The method comprises a) reacting minocycline and an hydroxymethylamide derivative to form a 2,9-(methylamide-substituted) minocycline and a 2-(methylamidesubstituted) minocycline; b) reacting the 2,9-(methylamide-substituted) minocycline from step a) and an amine or diamine to form a 9-aminomethyl tetracycline intermediate; and c) reacting the 9-aminomethyl tetracycline intermediate from step b) and an aldehyde in the presence of a reducing agent to form a 9-aminomethyl tetracycline compound; or d) reacting the 9-aminomethyl tetracycline intermediate from step b) and an alkyl halide or an alkyl reagent to form a 9-aminomethyl tetracycline compound. Step b) may be operated in the absence of a hydrogenation reaction. The method may be a semi continuous or continuous flow process.

### BACKGROUND OF THE INVENTION

The present invention relates to a new and improved method of producing 9-aminomethyl tetracycline compounds known from the prior art (U.S. Pat. No. 9,365,500 B2), including but not limited to omadacycline.

### DESCRIPTION OF THE PRIOR ART

Antibiotics are essential life-saving drugs that revolutionized medicine, starting with the discovery of penicillin in 1928 (Singh, S.; Barrett, J. Empirical Antibacterial Drug Discovery-Foundation in Natural Products. Biochem. Pharmacol. 2006, 71, 1006-1015). Since then, a number of highly effective antibiotics have been discovered and developed for clinical use in the treatment of bacterial infections (Brown, E.; Wright, G. Antibacterial Drug Discovery in the Resistance Era. Nature 2016, 529, 336-343). Many of these antibiotics have a broad-spectrum of activity, being effective in the treatment of infections caused by Gram-positive as well as Gram-negative bacteria, while others are effective only against Gram-positive bacteria. An ideal antibiotic is an antibacterial agent that kills or inhibits the growth of harmful bacteria in a host regardless of site of infection without affecting beneficial microbes (such as gut/skin flora). In any case, an antibiotic, ideal or not, does not remain an effective antibiotic forever mainly because of excessive or inappropriate prescribing, which has led to the increasing emergence and the spread of multi-resistant bacteria (Singh, S. B.; Young, K.; Silver, L. L. What Is an “Ideal” Antibiotic? Discovery Challenges and Path Forward. Biochem. Pharmacol. 2017, 133, 63-73).

Antimicrobial resistance (AMR) decreases our capability to treat infectious diseases and threatens our ability to perform routine surgery. As underlined in the EU One Health Action Plan on AMR (European Commission. A European One Health Action Plan against Antimicrobial Resistance (AMR); 2017) and the US government's report on antibiotic resistance threats (U.S Department of Health and Human Services, Antibiotic Resistance Threats in the United States—Report 2019; 2019) it is a problem of global concern with serious health and economic ramifications. An important challenge is the excessive and inappropriate use of antimicrobials in animal and human healthcare, leading to the development of resistance, causing an estimated 33,000 human deaths in the EU/EEA and more than 35,000 human deaths in the US every year (Cassini, A.; Högberg, L. D.; Plachouras, D.; Quattrocchi, A.; Hoxha, A.; Simonsen, G. S.; Colomb-Cotinat, M.; Kretzschmar, M. E.; Devleesschauwer, B.; Cecchini, M.; Ouakrim, D. A.; Oliveira, T. C.; Struelens, M. J.; Suetens, C.; Monnet, D. L. Attributable Deaths and Disability-Adjusted Life-Years Caused by Infections with Antibiotic-Resistant Bacteria in the EU and the European Economic Area in 2015: A Population-Level Modelling Analysis. Lancet 2019, 19, 56-66). It is estimated that nowadays a person dies every 1 minute and 23 seconds due to drug resistance infections and by 2050, the death toll could be a staggering one person every 3 seconds if the research and development of new drugs and therapies continues with this pace (O'Neill, J. Tackling Drug-Resistant Infections Globally: Final Report and Recommendations; 2016). Current incentive models do not provide a sustainable solution; new business approaches are required, including new incentives to develop antimicrobials as well as new pricing systems.

Investment in research and development for innovative medicines and treatments is essential for making progress in preventing and treating diseases. Access to safe, high quality and effective medicines is a key element of social well-being (European Commission. Pharmaceutical Strategy for Europe 2020; 2020). The continued development of new antibacterial agents is recognized to be very important for human health. In the face of increasing resistance, there is a need for new antibacterial agents suitable for treating infections in patients. Furthermore, in recent years there have been initiatives to re-evaluate dose regimens for some licensed agents to maximize their efficacy and minimize the risk of selecting resistant bacteria (EMEA Guideline on the Evaluation of Medicinal Products Indicated for Treatment of Bacterial Infections (Draft). EMEA Eur. Med. Agency 2010, 44 (February), 1-26). However, there have been few new antibiotics brought to market because of the lack of commercial interest (Wright, G. D. Perspective Antibiotics: A New Hope. Chem. Biol. 2012, 19 (1), 3-10). Currently, investment does not necessarily focus on the greatest unmet needs, due to the absence of commercial interest or limitations of the science. For example, there is a lack of development of new antimicrobials, treatments, or vaccines for emerging health threats (including those like the covid-19 pandemic, such as the severe acute respiratory syndrome coronavirus 2 (SARS-CoV-2) or Middle East respiratory syndrome (MERS)) and there is a lack of treatments for specific population groups such as pregnant and breastfeeding women and older people. Development of novel antimicrobials or alternatives is a prime example of unmet medical need, given the lack of therapeutic options to address AMR. The identification of new biochemical targets to make different classes of drugs is complex, and then there is the economic challenge associated with the low return on investment. Groundbreaking new antibiotics enter the market without resistance from bacteria, and thus the guideline is to reserve these drugs to be a last resort in cases where all other therapeutic options have failed, so the sales are marginal. To complement this issue, there are a number of antibiotics already in the clinic, including lower-cost generic drugs (Sertkaya, A.; Eyraud, J.; Birkenbach, A.; Franz, C.; Ackerley, N.; Overton, V. Analytical Framework for Examining the Value of Antibacterial Products.; Washington D. C., 2014).

In 2012, GAIN (Generating Antibiotic Incentives Now) was signed into law in the US as part of the Food and Drug Administration Safety and Innovation Act. Incentives were created for sponsors to bring to market antibacterial and antifungal drugs intended to treat serious or life-threatening infections (known as QIDPs-Qualified Infectious Disease Products). Sponsors may request GAIN for a drug, and FDA will review the request and respond within 60 days of submission (FDA Generating Antibiotics Incentive Now; 2017)

Sponsors who develop and submit applications for QIDPs may be eligible to receive incentives through GAIN. The primary incentive contained in GAIN is that designation as a QIDP qualifies the drug for 5 years of marketing exclusivity to be added to certain exclusivity already provided by the Food, Drug, and Cosmetic Act. GAIN also makes drug products that have been designated as QIDPs eligible for Fast Track designation. Finally, GAIN requires the FDA to give a priority review to the first application submitted for approval of a QIDP.

### Tetracyclines

Tetracyclines, a broad-spectrum class of antibiotics, are inhibitors of bacterial growth by inhibiting protein synthesis. In general, they bind to the bacterial 30S ribosomal subunit preventing the addition of amino acids to the growing polypeptide chain. (U.S. Pat. No. 9,365,500 B2, Bradford, P.; Jones, C. Antibiotic Discovery and Development—Chapter 5 (Tetracyclines); Springer, Boston, M A, 2012). Tetracyclines have been proven to be safe and effective over seven decades since the first tetracycline was discovered (Bradford, P.; Jones, C. Antibiotic Discovery and Development—Chapter 5 (Tetracyclines); Springer, Boston, M A, 2012).

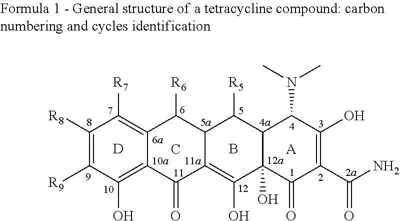

In 1945, Benjamin Duggar isolated 7-chlorotetracycline (compound 1, Formula 2) from a bacterial culture (Duggar, B. M. Aureomycin: A Product of the Continuing Search for New Antibiotics. Ann. N. Y. Acad. Sci. 1948, 51, 177-181) that was approved for human use in 1948 (Lederle Laboratories: https://www.accessdata.fda.gov/scripts/cder/daf/index.cfm?event=overview.process&ApplNo=050404). Two years later, 5-oxytetracycline (compound 2, Formula 2) was isolated (Finlay, A.; Hobby, G.; Regna, P.; Routien, J.; Seeley, D.; Shull, G. Terramycin, a New Antibiotic. Science (80) 1950, 11 (27), 85-85) by Pfizer scientists and it was approved by the FDA (Pfizer. https://www.accessdata.fda.gov/scripts/cder/daf/index.cfm?event=overview.process&ApplNo=050286) for human use (U.S Department of Health and Human Services. Antibiotic Resistance Threats in the United States—Report 2019; 2019). In 1953, Pfizer chemists modified 7-chlorotetracycline to produce the molecule known as tetracycline (compound 3, Formula 2Error! Reference source not found.) an even more active antibiotic (Bradford, P.; Jones, C. Antibiotic Discovery and Development—Chapter 5 (Tetracyclines); Springer, Boston, M A, 2012).

Lederle Laboratories led the discovery effort, while Pfizer, and more recently Paratek Pharmaceuticals and Tetraphase, have added value to the tetracycline class of antibiotics. Unfortunately, during the past decade, there has been a decline in efforts to discover and develop new and modified antibacterial agents so that they are not affected by common bacterial tetracycline resistance mechanisms (Bradford, P.; Jones, C. Antibiotic Discovery and Development—Chapter 5 (Tetracyclines); Springer, Boston, M A, 2012).

Significant class-based resistances due to the expression of tetracycline-specific efflux pumps and ribosome protection mechanisms have reduced the effectiveness of tetracyclines. In 1999, tigecycline (compound 4, Formula 2), a minocycline derivative in the glicylcycline subclass emerged as a broad-spectrum tetracycline that could avoid ribosomal protection and active efflux resistance mechanisms with activity against drug-resistant Gram-negative and Gram-positive organisms (Glycylcyclines: Third-Generation Tetracycline Antibiotics Ian Chopra. 2001, 464-469; Bush, K. Improving Known Classes of Antibiotics: An Optimistic Approach for the Future. Curr. Opin. Pharmacol. 2012, 12 (5), 527-534). In 2005, it was approved for the treatment of complicated skin and soft-tissue and complicated intra-abdominal infections (Babinchak, T.; Ellis-Grosse, E.; Dartois, N.; Rose, G.; Loh, E. The Efficacy and Safety of Tigecycline for the Treatment of Complicated Intra-Abdominal Infections: Analysis of Pooled Clinical Trial Data. Clin. Infect. Dis. 2005, 41 (s5), S354-S367). In 2008, it was approved by the FDA for the treatment of community-acquired bacterial respiratory infections (Stein, G. E.; Babinchak, T. Tigecycline: An Update. Diagn. Microbiol. Infect. Dis. 2013, 75 (4), 331-336). However, it is a drug administered intravenously only. It causes significantly more nausea and vomiting than other tetracyclines (Shen, F.; Han, Q.; Xie, D.; Fang, M.; Zeng, H.; Deng, Y.; Efficacy and Safety of Tigecycline for the Treatment of Severe Infectious Diseases: An Updated Meta-Analysis of RCTs. International Journal of Infectious Diseases. 2015, pp 25-33) and might cause mutations in Gram-negative efflux pumps during therapy (Pournaras, S.; Koumaki, V.; Spanakis, N.; Gennimata, V. Current Perspectives on Tigecycline Resistance in Enterobacteriaceae Susceptibility Testing Issues and Mechanisms of Resistance. Int. J. Antimicrob. Agents 2016, 48, 11-18). More recently, a new generation of tetracyclines came out to overcome the toxicity of tigecycline namely Omadacycline (5), Eravacycline (6) and Sarecycline (7)—Formula 2.

Omadacycline from Paratek Pharmaceuticals known as Nuzyra™ is a novel aminomethyl substituted derivative of minocycline (compound 8, Formula 2). Nuzyra™ was approved by the FDA in October 2018 (https://www.accessdata.fda.gov/scripts/cder/ob/results_product.cfm?Appl_Type=N&Appl_No=209817)—tablet form (NDA 209816) and powder form (NDA 209817)—for the treatment of community-acquired bacterial pneumonia (CABP) and acute bacterial skin and skin structure infections (ABSSSI) (Paratek Pharmaceuticals. Full Prescribing Information NUZYRA (omadacycline). Paratek Pharmaceuticals, Inc 2018).

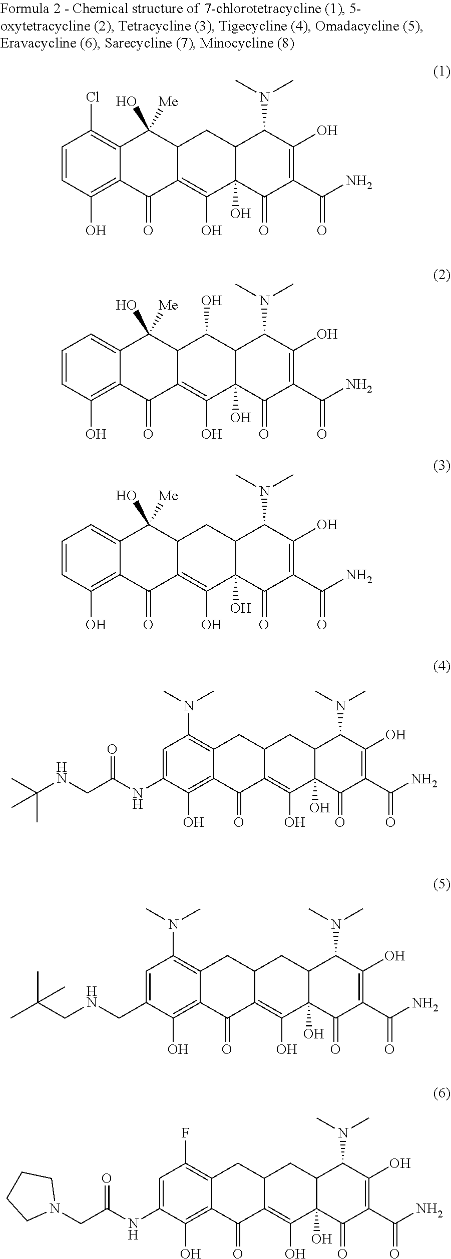

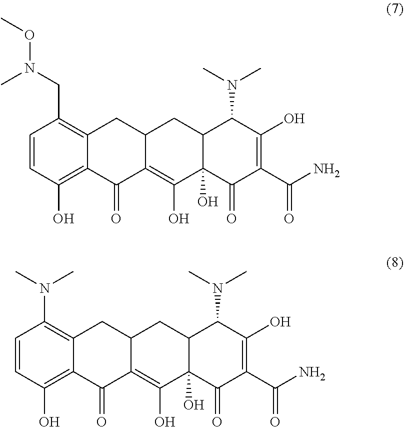 [SMILES: CON(C)Cc1ccc(O)c2c1CC1CC3[C@H](N(C)C)C(O)=C(C(N)=O)C(=O)[C@@]3(O)C(O)=C1C2=O ; CN(C)c1ccc(O)c2c1CC1CC3[C@H](N(C)C)C(O)=C(C(N)=O)C(=O)[C@@]3(O)C(O)=C1C2=O]

### Omadacycline

Omadacycline is a novel first-in-class aminomethylcycline and a semisynthetic derivative of minocycline (Honeyman, L.; Ismail, M.; Nelson, M. L.; Bhatia, B.; Bowser, T. E.; Chen, J.; Mechiche, R.; Ohemeng, K.; Verma, A. K.; Cannon, E. P.; Macone, A.; Tanaka, S. K.; Levy, S. Structure-Activity Relationship of the Aminomethylcyclines and the Discovery of omadacycline. Antimicrob. Agents Chemother. 2015, 59 (11), 7044-7053). It is characterized by an aminomethyl substituent at the C9 position of the tetracycline D ring according to Formula 1 (U.S. Pat. No. 9,365,500 B2). Modifications at this position resulted in an enhanced activity against Gram-positive and Gram-Negative bacteria overcoming resistance mechanisms known to affect older generation tetracyclines (i.e. efflux and ribosomal protection) (Chopra, I.; Roberts, M. Tetracycline Antibiotics: Mode of Action, Applications, Molecular Biology, and Epidemiology of Bacterial Resistance. Microbiol. Mol. Biol. Rev. 2001, 65 (2), 232-260; Gotfried, M. H.; Horn, K.; Garrity-Ryan, L.; Villano, S.; Tzanis, E.; Chitra, S.; Manley, A.; Tanaka, S. K.; Rodvoldb, K. A. Comparison of omadacycline and Tigecycline Pharmacokinetics in the Plasma, Epithelial Lining Fluid, and Alveolar Cells of Healthy Adult Subjects. Antimicrob. Agents Chemother. 2017, 61 (9), 1-13).

The primary effect of omadacycline is on bacterial protein synthesis inhibition with great potency. (Draper, M. P.; Weir, S.; Macone, A.; Donatelli, J.; Trieber, C. A.; Tanaka, S. K.; Levy, S. B. Mechanism of Action of the Novel Aminomethylcycline Antibiotic omadacycline. Antimicrob. Agents Chemother. 2014, 58 (3), 1279-1283). It acts by binding to the 30S ribosomal subunit in the mRNA translation complex of bacteria and inhibiting the binding of aminoacyl-tRNA to the mRNA-ribosome complex, consequently, inhibiting the expression of proteins. (Gotfried, M. H.; Horn, K.; Garrity-Ryan, L.; Villano, S.; Tzanis, E.; Chitra, S.; Manley, A.; Tanaka, S. K.; Rodvoldb, K. A. Comparison of omadacycline and Tigecycline Pharmacokinetics in the Plasma, Epithelial Lining Fluid, and Alveolar Cells of Healthy Adult Subjects. Antimicrob. Agents Chemother. 2017, 61 (9), 1-13).

In January 2013, the FDA has designated omadacycline as a QIDP for both IV and oral formulations in the treatment of acute bacterial skin and skin structure infections (ABSSSI) and community-acquired bacterial pneumonia (CABP) (Liapikou, A.; Cilloniz, C.; Mensa, J.; Torres, A. Pulmonary Pharmacology & Therapeutics New Antimicrobial Approaches to Gram Positive Respiratory Infections. Pulm. Pharmacol. Ther. 2015, 32, 137-143; Berg, J. K.; Tzanis, E.; Garrity-Ryan, L.; Bai, S.; Chitra, S.; Manley, A.; Villano, S. Pharmacokinetics and Safety of omadacycline in Subjects with Impaired Renal Function. Antimicrob. Agents Chemother. 2018, 62 (2), 1-9). ABSSSI includes cellulitis/erysipelas, wound infection, and major cutaneous abscesses. (FDA Acute Bacterial Skin and Skin Structure Infections: Developing Drugs for Treatment; 2013). CABP is a common disease in adults with 5.16 to 7.06 cases per 1000 persons per year (Marrie, T. J.; Huang, J. Q. Epidemiology of Community-Acquired Pneumonia in Edmonton, Alberta: An Emergency Department-Based Study. Can. Respir. J. 2005, 12 (3), 139-143).

In October 2018, omadacycline received FDA approval (Paratek Pharmaceuticals. Full Prescribing Information NUZYRA (omadacycline). Paratek Pharmaceuticals, Inc 2018; FDA Omadacycline Injection and Oral Products https://www.fda.gov/drugs/development-resources/omadacycline-injection-and-oral-products (accessed Mar. 1, 2021)) for the treatment of patients with serious skin and soft tissue infections and community-acquired bacterial pneumonia (CAPB). (O'Riordan, W.; Green, S.; Overcash, J. S.; Puljiz, I.; Metallidis, S.; Gardovskis, J.; Garrity-Ryan, L.; Das, A. F.; Tzanis, E.; Eckburg, P. B.; Manley, A.; Villano, S. A.; Steenbergen, J. N.; Loh, E. omadacycline for Acute Bacterial Skin and Skin-Structure Infections. N. Engl. J. Med. 2019, 380 (6), 528-538; Stets, R.; Popescu, M.; Gonong, J. R.; Mitha, I.; Nseir, W.; Madej, A.; Kirsch, C.; Das, A. F.; Garrity-Ryan, L.; Steenbergen, J. N.; Manley, A.; Eckburg, P. B.; Tzanis, E.; McGovern, P. C.; Loh, E. omadacycline for Community-Acquired Bacterial Pneumonia. N. Engl. J. Med. 2019, 380 (6), 517-527; Chopra, T.; Sandhu, A.; Theriault, N.; Meehan, J.; Tillotson, G. omadacycline: A Therapeutic Review of Use in Community-Acquired Bacterial Pneumonia and Acute Bacterial Skin and Skin Structure Infections. Future Microbiol. 2020, 15 (14), 1319-1333; Lakota, E. A.; Van Wart, S. A.; Trang, M.; Tzanis, E.; Bhavnani, S. M.; Safir, M. C.; Friedrich, L.; Steenbergen, J. N.; Ambrose, P. G.; Rubino, C. M. Population Pharmacokinetic Analyses for omadacycline Using Phase 1 and 3 Data. Antimicrob. Agents Chemother. 2020, 64 (7), 1-10; US 2018/0153908 A1).

Omadacycline can be administered intravenously or orally. Its broad spectrum of activity is superior to doxycycline (Draper, M. P.; Weir, S.; Macone, A.; Donatelli, J.; Trieber, C. A.; Tanaka, S. K.; Levy, S. B. Mechanism of Action of the Novel Aminomethylcycline Antibiotic omadacycline. Antimicrob. Agents Chemother. 2014, 58 (3), 1279-1283), minocycline, clindamycin, linezolid (O'Riordan, W.; Cardenas, C.; Shin, E.; Sirbu, A.; Garrity-Ryan, L.; Das, A. F.; Eckburg, P. B.; Manley, A.; Steenbergen, J. N.; Tzanis, E.; McGovern, P. C.; Loh, E. Once-Daily Oral omadacycline versus Twice-Daily Oral Linezolid for Acute Bacterial Skin and Skin Structure Infections (OASIS-2): A Phase 3, Double-Blind, Multicentre, Randomised, Controlled, Non-Inferiority Trial. Lancet Infect. Dis. 2019, 19 (10), 1080-1090; Noel, G. J.; Draper, M. P.; Hait, H.; Tanaka, S. K.; Arbeit, R. D. A Randomized, Evaluator-Blind, Phase 2 Study Comparing the Safety and Efficacy of omadacycline to Those of Linezolid for Treatment of Complicated Skin and Skin Structure Infections. 2012, 56 (11), 5650-5654) or vancomycin. (Macone, A. B.; Caruso, B. K.; Leahy, R. G.; Donatelli, J.; Weir, S.; Draper, M. P.; Tanaka, S. K.; Levy, S. B. In Vitro and in Vivo Antibacterial Activities of omadacycline, a Novel Aminomethylcycline. Antimicrob. Agents Chemother. 2014, 58 (2), 1127-1135). It has potent bacteriostatic activity against Gram-positive Methicillin Resistant Staphylococcus Aureus (MRSA), multidrug-resistant S. pneumonia, vancomycin-resistant enterococci, E. faecalis or E. faecium, and S. pneumoniae strains including penicillin and multi-resistant strains and anaerobic Clostridium difficille (WO2017/165729A1, 2016). Also against Gram-Negative Haemophilus influenzae, E. coli and Legionella (Huband, M. D.; Pfaller, M. A.; Shortridge, D.; Flamm, R. K. Surveillance of omadacycline Activity Tested against Clinical Isolates from the United States and Europe: Results from the SENTRY Antimicrobial Surveillance Programme, 2017. J. Glob. Antimicrob. Resist. 2019, 19, 56-63; U.S. Pat. No. 9,724,358 B2). Consequently, omadacycline may be an important and desirable treatment alternative for patients with infections where the epidemiology suggests a problematic prevalence of resistant pathogens (Macone, A. B.; Caruso, B. K.; Leahy, R. G.; Donatelli, J.; Weir, S.; Draper, M. P.; Tanaka, S. K.; Levy, S. B. In Vitro and in Vivo Antibacterial Activities of omadacycline, a Novel Aminomethylcycline. Antimicrob. Agents Chemother. 2014, 58 (2), 1127-1135). Methods of treatment using omadacycline are also described for urinary tract infections (UTIs), bacterial infections caused by a biological weapon and for regulating expression of genes (U.S. Pat. No. 9,724,358 B2; Bal, A. M.; David, M. Z.; Garau, J.; Gottlieb, T.; Mazzei, T.; Scaglione, F.; Tattevin, P.; Gould, I. M. Future Trends in the Treatment of Methicillin-Resistant Staphylococcus Aureus (MRSA) Infection: An in-Depth Review of Newer Antibiotics Active against an Enduring Pathogen. J. Glob. Antimicrob. Resist. 2017, 10, 295-303; WO 2017/192516 A1; WO 2016/154332 A1; WO 2007/133798 A1; WO 2018/026987 A1; U.S. Pat. No. 9,078,811 B2; WO 2009/120389 A1). Phase 1 clinical trials are ongoing for tissue penetration in diabetic patients with wound infections via hemodialysis (NCT04144374) and for the treatment of diabetic foot infections (NCT04714411). Phase 2 clinical trials are complete for the treatment of acute pyelonephritis in adults (NCT03757234) and oral treatment of acute cystitis in women (NCT03425396) (Overcash, J. S.; Bhiwandi, P.; Garrity-ryan, L.; Steenbergen, J.; Bai, S.; Chitra, S.; Manley, A.; Tzanis, E. Pharmacokinetics, Safety, and Clinical Outcomes of omadacycline in Women with Cystitis: Results from a Phase 1b Study. Antimicrob. Agents Chemother. 2019, 63 (5), 1-10).

In 2019, Paratek Pharmaceuticals Inc. was awarded with a BARDA Project BioShield 5-year contract valued up to $285 million to support the development of Paratek's NUZYRA® (omadacycline) for the treatment of pulmonary anthrax, and the option to procure up to 10,000 treatment courses of NUZYRA® for the Strategic National Stockpile (SNS) for use against potential biothreats (Paratek Pharmaceuticals. Paratek Awarded BARDA Project BioShield Contract for NUZYRA® https://www.globenewswire.com/news-release/2019/12/18/1962517/0/en/Paratek-Awarded-BARDA-Project-BioShield-Contract-for-NUZYRA.html (accessed Mar. 5, 2021)).

The current synthesis of omadacycline, depicted in Scheme 1, has been used for multi-kilogram preparations and is described in U.S. Pat. No. 9,434,680 B2. (9-aminomethyl tetracycline compounds other than omadacycline are also disclosed in U.S. Pat. No. 9,434,680 B2).

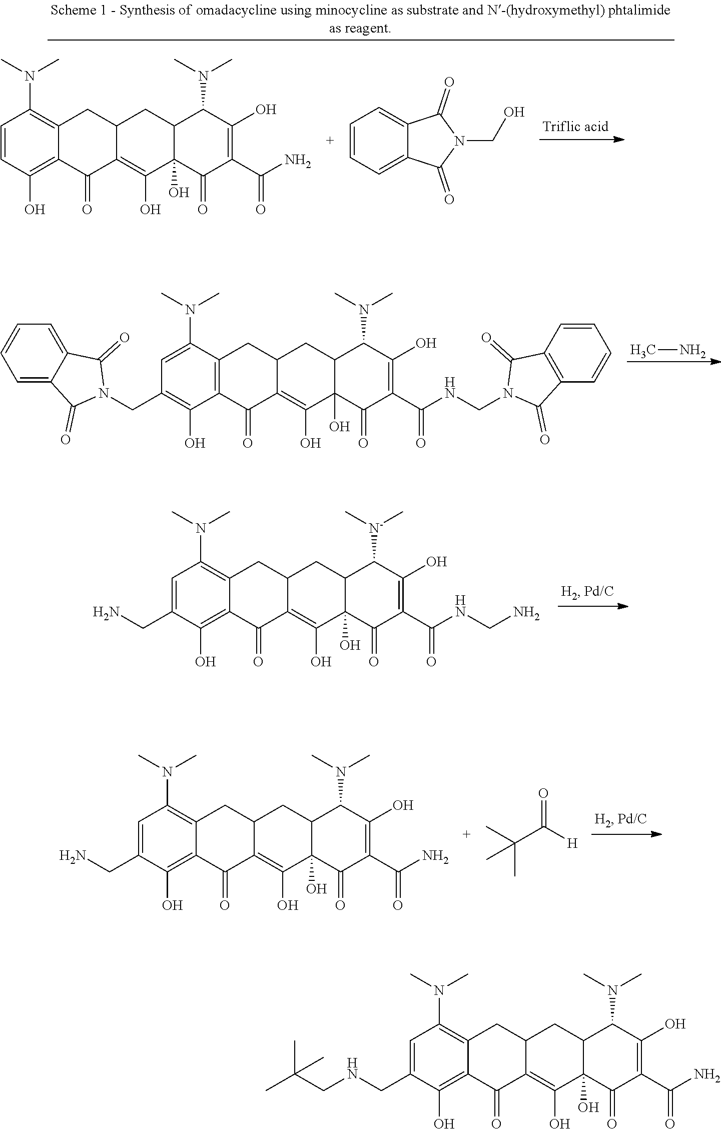

Minocycline has several reactive functional groups and the C2 primary amide is more reactive towards electrophiles than C9 or the C10 (Formula 1). Due to this fact, the first step of Scheme 1 requires nearly three equivalents of the N′-(hydroxymethyl)-phthalimide in triflic acid yielding a bis-substituted aminomethyl-phtalimide tetracycline compound. In the second step of Scheme 1, the phthalimides are de-protected with a large excess of methylamine in alcoholic solution to afford a bis-substituted aminomethyl tetracycline intermediate. In the third step of Scheme 1, the resulting intermediate is reacted with hydrogen under hydrogenation conditions to form a C9-substituted aminomethyl tetracycline intermediate. In the fourth step of Scheme 1, the formed compound is reacted with pivaldehyde under hydrogenation conditions to afford omadacycline. After reverse-phase chromatographic purification, pH adjustment and precipitation the desired product is afforded as an amorphous, unstable solid. For a long-term manufacturing route, the challenges of the instability of the aminomethyl intermediate and chromatographic column purification step must be overcome.

A three-step synthesis of omadacycline is described via an electronically tuned mono chloro acyliminium Friedel Crafts reaction (Tscherniac-Einhorn reaction) using an acyliminium ion as starting material with 15-18% overall yield (see Scheme 2 and Chung, J. Y. L.; Hartner, F. W.; Cvetovich, R. J. Synthesis Development of an Aminomethylcycline Antibiotic via an Electronically Tuned Acyliminium Friedel-Crafts Reaction. Tetrahedron Lett. 2008, 49 (42), 6095-6100).

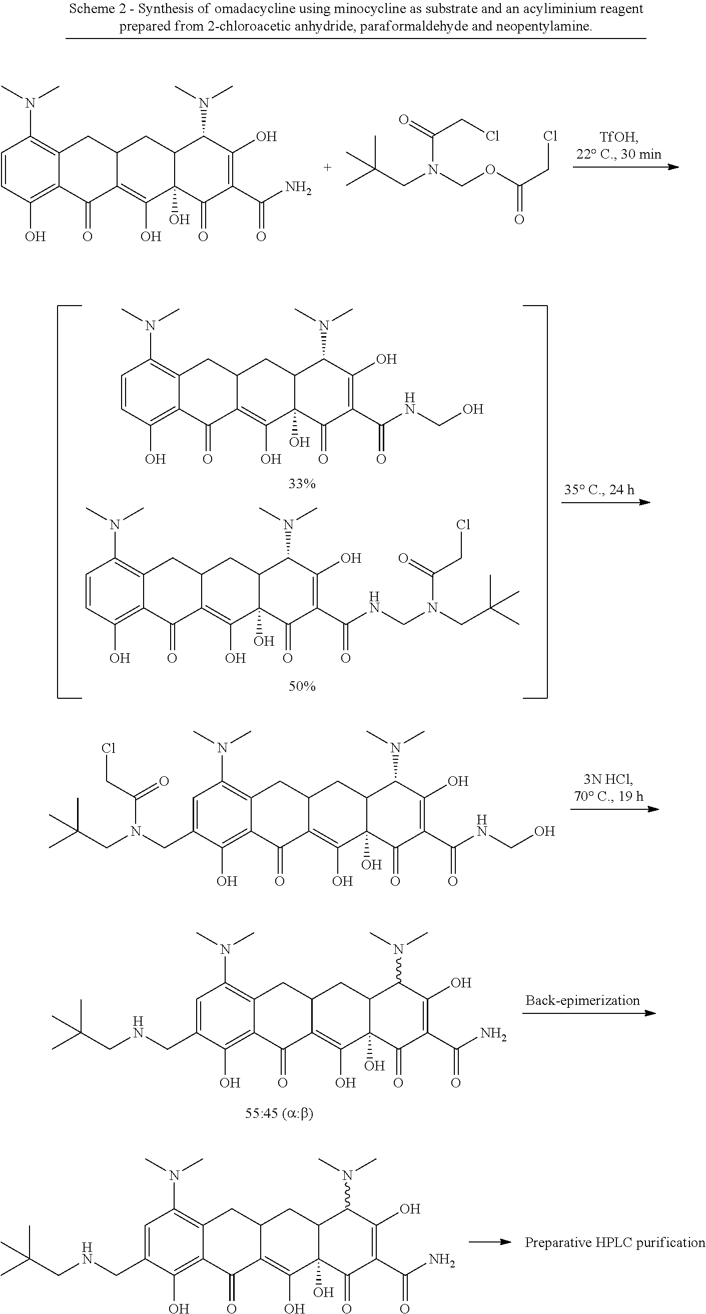

The acyliminium reagent is prepared from neopentylamine and paraformaldehyde to afford triazane, followed by the treatment with anhydrides (Chung, J. Y. L.; Hartner, F. W.; Cvetovich, R. J. Synthesis Development of an Aminomethylcycline Antibiotic via an Electronically Tuned Acyliminium Friedel-Crafts Reaction. Tetrahedron Lett. 2008, 49 (42), 6095-6100; Taguchi, M.; Aikawa, N.; Tsukamoto, G. Reaction of Rifamycin S with Hexahydro-1,3,5-Triazines Prepared from Formaldehyde and Primary Aliphatic Amines. Bull Chem Soc Jpn 1988, 61, 2431-2436; Anderson, J.; Casarini, D.; Ijeh, A. Eclipsed Conformation for Both Axial and Equatorial N—CH2 Bonds in N,N′,N″-Tris(Neopentyl)-1,3,5-Triazane. J. Am. Chem. Soc. 1995, 117 (11), 3054-3056). Triflic acid is the solvent used because according to Chung J. et. al, solutions of minocycline in triflic acid are stable to air oxidation, C4 epimerization and other ways of degradation. In the first step of Scheme 2, the optimized yield is 83% using 5 equivalents of acyliminium at 35/40° C. for 24 hours. The second step consists in the removal of the chloro-acetyl group, heating the chloro-acetyl intermediate in 3N HCl at 70° C. for 20 hours. Back-epimerization is performed heating the racemic mixture of omadacycline (crude) at 105° C. in aqueous n-butanol in the presence of calcium chloride and ethanolamine. The resultant amorphous solid is unstable at temperatures above 0° C. and when exposed to air, thus it is necessary to prepare a stable salt. The crystalline salts of omadacycline (mono-tosylate, bis HCl and mesylate) are stable at 25° C. (US 2018/0104262 A1). However, in this synthesis, the purification step must be changed from chromatographic systems to kilogram scale feasible operations.

U.S. Pat. No. 9,365,500 B2 described a synthetic route for omadacycline based on the Tscherniac-Einhorn reaction and is described in Scheme 3. In the first step of Scheme 3, the aminomethyl intermediate can be synthesized using N′-(hydroxymethyl) benzyl carbamate in acidic medium at 25° C. for 24 hours using minocycline as substrate. The second step consists of the reductive amination of the intermediate yielding Omadacycline (U.S. Pat. No. 9,365,500 B2).

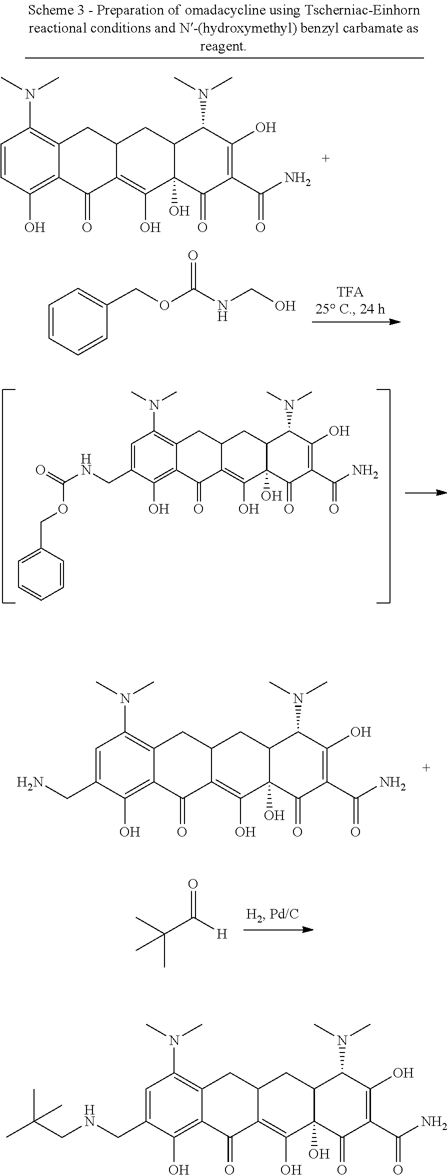

In 1983, Baillargeon et al (Baillargeon, V.; Stille, J. Direct Conversion of Organic Halides to Aldehydes with Carbon Monoxide and Tin Hydride Catalyzed by Palladium. J. Am. Chem. Soc. 1983, 105, 7175) described a palladium-catalyzed formylation of organic-halide substrates in the presence of carbon monoxide giving aldehydes in good yields. Seyedi F. et al. described in U.S. Pat. No. 9,522,872 B2 this procedure for the preparation of 9-iodo minocycline and subsequently of 9-formyl minocycline in 99% yield. The resultant intermediate is reacted with sodium triacetoxyborohydride to afford omadacycline.

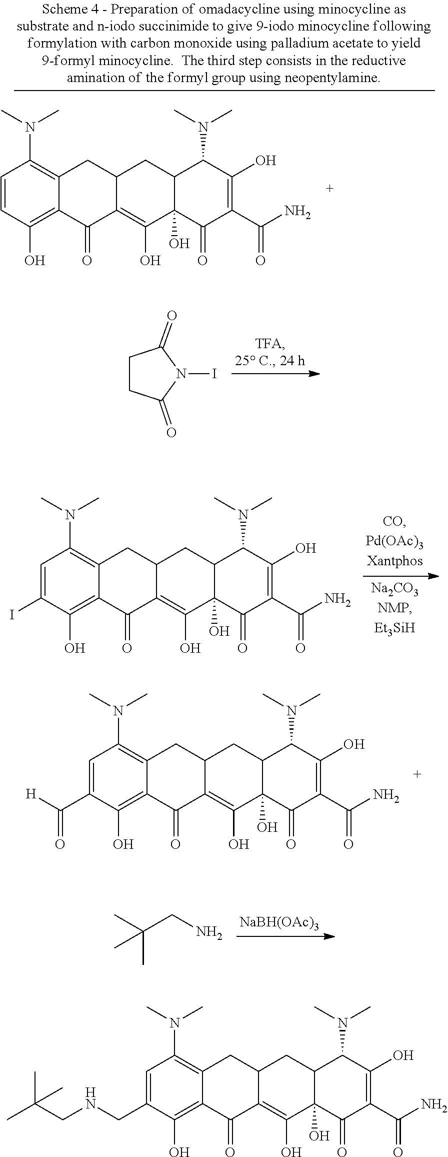

Omadacycline has proved to be safe and to be greatly effective against multidrug-resistant Gram-positive and Gram-negative bacteria but, as far as the inventors know, all described methods for synthesizing 9-aminomethyl tetracycline compounds present low yields, low selectivity and great difficulties in the purification steps. For an industrial scale synthetic method there is still a need for optimization to achieve better yields with reduced impurities when preparing this type of compound. This invention provides a new method using innovative chemical strategies. Making use of continuous flow technologies, the inventors took advantage of process intensification (e.g. high temperatures and pressure) and improved mass and heat transfer to increase selectivity and overcome the challenges of purification, low yields and the isolation of an unstable intermediate. Moreover, keeping the concepts of green chemistry in mind, the inventors developed a method choosing solvents and reagents that are environmentally friendly whenever possible.

The inventors' sought to develop new, greener, cheaper, and better methods for synthesizing 9-aminomethyl tetracycline compounds, especially omadacycline, which methods can ultimately benefit patients, companies, and the environment. The present invention meets such goals since it provides a method which reduces or eliminates one or more of the problems with the known methods as outlined above.

According to the present invention, the inventors developed a method that may be operated as a semi-continuous or continuous process, which method is used to prepare amino-alkyl tetracycline compounds such as 9-aminomethyl minocycline in as ecologically clean a way as possible, reducing time and waste. The inventors have found that it is possible to use environmentally friendly solvents, avoid degradation of sensitive intermediates and control the formation of epimers by using flow chemistry technologies, which makes it possible to obtain aminomethyl tetracycline compounds with purity above 50%, preferably between 70 and 80% and more preferably between 81 and 100%. Moreover, global yields (otherwise known as accumulated yields) above 30%, preferably between 50 and 70%, and more preferably between 71 and 100%. One example of an aminomethyl tetracycline compound that can be prepared by the method of the present invention is omadacycline.

### DESCRIPTION OF THE INVENTION

According to one aspect of the present invention, there is provided a method for synthesizing 9-aminomethyl tetracycline compounds according to Formula 3, wherein R is a hydrogen or a C1 to C10 straight chain alkyl group, a C3-C20 branched chain alkyl group, a substituted alkyl group (optionally substituted with at least one of halogens, hydroxyl groups, ketones and ethers), a C3 to C10 or C6 to C10 aryl group, a substituted C3 to C10 or C6 to C10 aryl group (optionally substituted with at least one of halogens, hydroxyl groups, ketones and ethers) or a C3 to C10 heteroaryl group comprising at least one oxygen, nitrogen, sulfur or phosphorous atom

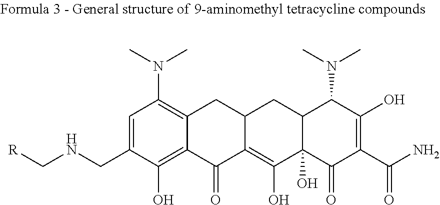

the method comprising:
a) reacting minocycline and an hydroxymethylamide derivative to form a 2,9-(methylamide-substituted) minocycline and a 2-(methylamide-substituted) minocycline;
b) reacting the 2,9-(methylamide-substituted) minocycline from step a) and an amine or diamine to form a 9-aminomethyl tetracycline intermediate; and
c) reacting the 9-aminomethyl tetracycline intermediate from step b) and an aldehyde in the presence of a reducing agent to form a 9-aminomethyl tetracycline compound; or
d) reacting the 9-aminomethyl tetracycline intermediate from step b) and an alkyl halide or an alkyl reagent to form a 9-aminomethyl tetracycline compound.

The method of the present invention is a multi-step method that uses an electrophilic aromatic substitution between minocycline and an hydroxymethylamide derivative in step a) to afford a 2,9-(methylamide-substituted) minocycline, an aminolysis reaction between the 2,9-(methylamide-substituted) minocycline and an amine or diamine in step b) to afford a 9-aminomethyl tetracycline intermediate, and either a reductive amination reaction between the 9-aminomethyl intermediate with an aldehyde, a reducing agent in step c) or N′-alkylation between the 9-aminomethyl intermediate and an alkyl halide or an alkyl reagent in step d) to form the desired 9-aminomethyl tetracycline compound. The term “multi-step chemical synthesis” as used herein generally relates to a synthetic method comprising multiple chemical reactions. The term is not intended to cover a synthetic method wherein merely one chemical reaction may be carried out over multiple steps.

The hydroxymethylamide derivative used in step a) may be in accordance with Formula 4

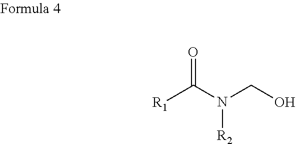

wherein R₁ is a C1-C10 straight chain alkyl group, a C3-C20 branched chain alkyl group, a C2-C10 straight chain alkenyl group, a C3-C20 branched chain alkenyl group, a C2-C10 straight chain alkynyl group, a C3-C20 branched chain alkynyl group, a C3 to C10 or C6 to C10 aryl group, a C3 to C10 heteroaryl group comprising at least one oxygen, nitrogen, sulfur or phosphorous atom, or an halogen selected from chlorine, bromine and iodine; and R₂ is a C1-C10 straight chain alkyl group, a C3-C20 branched chain alkyl group, a C2-C10 straight chain alkenyl group, a C3-C20 branched chain alkenyl group, a C2-C10 straight chain alkynyl group, a C3-C20 branched chain alkynyl group, a C3 to C10 or C6 to C10 aryl group, or a C3 to C10 heteroaryl group comprising at least one oxygen, nitrogen, sulfur or phosphorous atom. R₂ is optionally linked to R₁ to form a 4-8 membered ring, wherein the ring may optionally be substituted with other functional groups, such as halogens, hydroxyl groups, ketones, ethers, esters and amides, and comprise carbon atoms and/or heteroatoms, such as oxygen, nitrogen, and sulfur. Optionally, the hydroxymethylamide derivative in step a) is N′-hydroxymethyl-phthalimide.

The term alkyl as used herein is a general term that refers to a group derived from an alkane by removal of a hydrogen atom from any carbon atom of the alkane, it includes saturated aliphatic groups, including straight-chain alkyl groups (e.g., methyl, ethyl, propyl, butyl, pentyl, etc.), branched-chain alkyl groups (e.g., isopropyl, tert-butyl, isobutyl, etc.), and cycloalkyl groups (e.g., cyclopropyl, cyclopentyl,etc). The term alkyl further includes alkyl groups, which can further include oxygen, nitrogen, sulfur or phosphorous atoms.

The term aryl as used herein is a general term that refers to any aromatic group derived from an arene (otherwise known as an aromatic hydrocarbon) by removal of a hydrogen atom from any carbon atom of an aromatic ring.

The amine or diamine used in step b) may be in accordance with Formula 5

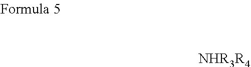

wherein R₃ and R₄ is a hydrogen atom, a C1-C10 straight chain alkyl group, a C3-C20 branched chain alkyl group, or a substituted alkyl group (optionally substituted with alcohols or ethers). Preferably R₃ and R₄ are selected from a C1-C4 straight chain alkyl group, a C3-C4 branched chain alkyl group, or a substituted alkyl group. Optionally, the amine or diamine in step b) is selected from methylamine, ethanolamine and n-propylamine.

An excess of amine or diamine may be used in step b). Optionally, the excess of amine or diamine may be continuously removed prior to step c) or d).

In contrast to some prior art methods, step b) may be operated in the absence of a hydrogenation reaction. That is, reacting the 2,9-(methylamide-substituted) minocycline and an amine or diamine directly forms a 9-aminomethyl tetracycline intermediate without the need to carry out a hydrogenation reaction on a compound to form the intermediate.

The aldehyde used in step c) may be in accordance with Formula 6

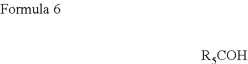

wherein R₅ is a hydrogen, a C1-C10 straight chain alkyl group, a C3-C20 branched chain alkyl group, a substituted alkyl group (optionally substituted with alcohols, amides or ethers), a C3 to C10 or C6 to C10 aryl group, a substituted C3 to C10 or C6 to C10 aryl group or a C3 to C10 heteroaryl group comprising at least one oxygen, nitrogen, sulfur or phosphorous atom. Optionally, the aldehyde used in step c) is selected from pivaldehyde, acetaldehyde and benzaldehyde.

The reducing agent used in step c) may be an immobilized reducing agent, optionally immobilized sodium cyanoborohydride.

Where an alkyl halide is used in step d), the alkyl halide may be in accordance with Formula 7

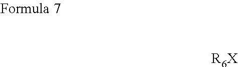

wherein R₆ can be a C1-C10 straight chain alkyl group, a C3-C20 branched chain alkyl group, a substituted alkyl group, a C3 to C10 or C6 to C10 aryl group, a substituted C3 to C10 or C6 to C10 aryl group or a C3 to C10 heteroaryl group; comprising at least one of oxygen, nitrogen, sulfur or phosphorous atom and X is an halogen selected from chlorine, bromine and iodine. Optionally, the alkyl halide used in step d) comprises 1-chloro-2,2-dimethylpropane, 1-bromo-2,2-dimethylpropane or 1-iodo-2,2-dimethylpropane.

Where an alkyl reagent is used in step d), the alkyl reagent may have a good leaving group, such as mesyl or tosyl. Optionally, the alkyl reagent is neopentyl 4-methylbenzenesulfonate, neopentyl methnesulfonate or mixtures thereof.

The reaction in step c) or d) may be carried out in the presence of a proton acceptor, optionally a proton acceptor selected from triethylamine, ammonia and 4-dimethylaminopyridine.

The reaction in step c) may be carried out in the presence of an organic acid, such as formic acid or acetic acid, an inorganic acid or mixtures thereof.

The ratio of reactants used in each of steps a), b) and c) or d) can vary from 1:1 to 1:30.

A method can be defined as a continuous flow process when there is a continuous feed of reagents/starting materials into a reactor with a continuous product stream exiting the reactor. Continuous flow processes make use of equipment, materials and conditions that allow chemical syntheses to be carried out in a continuous mode using flow reactors. A continuous flow procedure herein used does not comprise the traditional procedure of chemical synthesis in batch.

The method of the present invention may be a semi continuous or continuous flow process. Hence, in a continuous flow process the whole synthetic sequence of the method of the present invention may be carried out from the minocycline reacted in step a) to the 9-aminomethyl tetracycline compound formed in step d) without the use of batch reactors, without the need to isolate the 9-aminomethyl tetracycline intermediate formed in step b) and in the absence of a hydrogenation reaction prior to step c) or d). Alternatively, in a semi continuous flow process, two of steps a), b) and c) or d) may be carried without the use of batch reactors, without the need to isolate intermediate products between the reaction steps. The steps a) and b) of the method of the present invention may operate in a continuous manner, steps b) and c) of the method of the present invention may operate in a continuous manner, or steps b) and d) of the method of the present invention may operate in a continuous manner. Where steps b) and c) or steps b) and d) operate in a continuous manner, the 9-aminomethyl tetracycline intermediate formed in step b) may be used directly in step c) or d).

In a semi continuous flow process some but not all of the reaction steps of the present invention may be carried out in continuous flow reactors. In a continuous flow process all the reactions steps of the present invention may be carried out in a single continuous flow reactor or in multiple continuous flow reactors in fluid communication with each other.

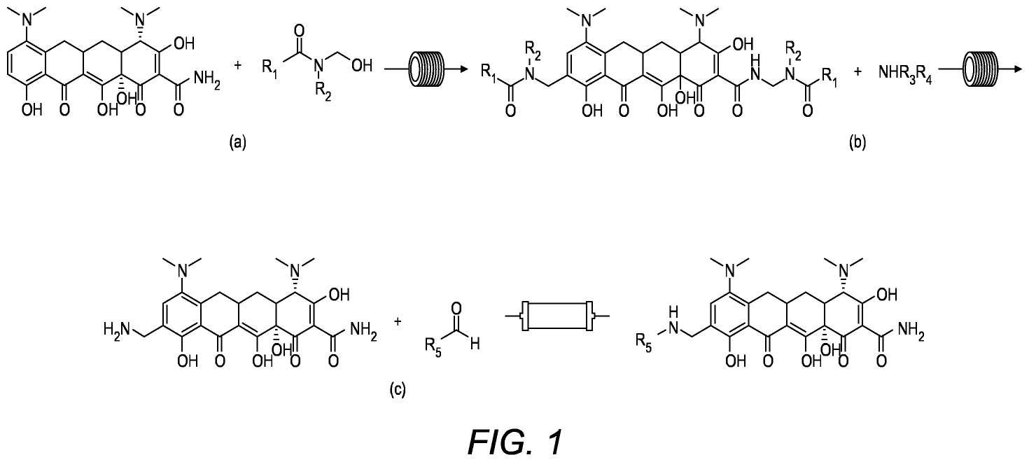

Step a) of the method of the present invention may comprise continuously feeding a solution or suspension comprising minocycline and a solution or suspension comprising hydroxymethylamide derivative in a suitable solvent or mixture of solvents to a flow reactor that continuously produces a solution or suspension comprising variable amounts of a 2-(methylamide-substituted) minocycline compound at the outlet. Step b) of the method of the present invention may comprise feeding a solution or suspension of the 2,9-(methylamide-substituted) minocycline compound and a solution or suspension comprising an amine or diamine in a suitable solvent to a flow reactor that continuously produces a solution or suspension comprising variable amounts of a 9-aminomethyl tetracycline intermediate at the outlet. Step c) of the method of the present invention may comprise feeding a solution or suspension of the 9-aminomethyl tetracycline intermediate, a solution or suspension of an aldehyde in a suitable solvent to a flow reactor containing a reducing agent that continuously produces a solution or suspension containing the desired 9-aminomethyl tetracycline compound at the outlet. Alternatively, step d) of the method of the present invention may comprise feeding a solution or suspension of the 9-aminomethyl tetracycline intermediate, a solution or suspension of an alkyl halide or alkyl reagent in a suitable solvent to a flow reactor that continuously produces a solution or suspension containing the desired 9-aminomethyl tetracycline compound at the outlet. Omadacycline is an antibiotic that may be produced by the synthetic sequence shown in FIG. 1.

Surprisingly, it has been found that it is possible to synthesize a 9-aminomethyl tetracycline intermediate directly from 2,9-(methylamide-substituted) minocycline compound by using higher temperatures that are only feasible by using flow chemistry technologies at low residence time.

The residence time of the reactions in steps a), b) and c) or d) may be from 12 seconds to 2 hours, optionally from 12 seconds to 30 minutes. The residence time of each reaction step may differ from the residence time of the other reaction steps.

The residence time of reagents along a selected distance of the continuous flow reactor which is associated with the electrophilic aromatic substitution reaction in step a), the aminolysis reaction in step b), the reductive amination reaction in step c) and the N′-alkylation in step d) can vary from 1 minute to 2 hours. The yield of each reaction step may be about 5% or more, preferably 50% or more, and more preferably 80% or more. The chromatographic purity of the resultant reaction crude from step a) or b) may be about 50% or more and more preferably 80% or more.

The solvents used in the method of the present invention may be common organic solvents, aqueous solvents, aqueous based solvents, water or mixtures thereof. Any compatible solvent or solvent system can be used. The solvent systems used may comprise colloidal suspensions or emulsions. The solvent systems used may comprise alcohols, water, or a mixture of both. The solvent systems may comprise mixtures of water-miscible organic solvents and water. They may also comprise water immiscible organic solvents in contact with or not in contact with water. Any specific combinations of the above listed solvents may be used. The method steps of the present invention may not be optimally carried out in the same solvent or solvent system and when, and if necessary, adjustment of the solvent/solvent composition or a solvent switch may be carried out in a continuous manner—for example, without the need to isolate or purify intermediates.

The minocycline used in step a) may be in solution or suspension, optionally wherein the solution or suspension comprises a solvent selected from an organic acid or mineral acid such as sulfuric acid, methanesulfonic acid, triflic acid, sulfuric acid fuming 65% SO₃ or mixtures thereof. Optionally, a solution or suspension of minocycline in sulfuric acid at a concentration of from 130 to 230 mg/mL may be used in step a). The hydroxymethylamide derivative used in step a) may be in solution or suspension, optionally wherein the solution or suspension comprises a solvent selected from an organic acid or mineral acid such as sulfuric acid, methanesulfonic acid, triflic acid, sulfuric acid fuming 65% SO₃ or mixtures thereof. Optionally, a solution or suspension of hydroxymethylamide derivative in sulfuric acid at a concentration of from 100 to 160 mg/mL may be used in step a). Therefore, the minocycline and the hydroxymethylamide derivative may be reacted together when both in solution, when one is in solution and the other is in suspension or when both are in suspension. In addition, the minocycline and the hydroxymethylamide derivative may be in solutions or suspensions comprising the same solvent or mixtures of solvents, or different solvents, or different combinations of solvents. Furthermore, the minocycline and the hydroxymethylamide derivative may be in solutions or suspensions at the same or differing concentrations.

The 2,9-(methylamide-substituted) minocycline used in step b) may be in solution or suspension, optionally wherein the solution or suspension comprises a solvent selected from an alcohol, such as benzyl alcohol, a polar aprotic solvent, such as dimethylsulfoxide, dimethylformamide or dichloromethane, or mixtures thereof. Optionally, a solution or suspension of 2,9-(methylamide-substituted) minocycline at a concentration of from 50 to 200 mg/mL may be used in step b). The amine or diamine used in step b) may be in solution or suspension, optionally wherein the solution or suspension comprises a solvent selected from an alcohol, such as benzyl alcohol, a polar aprotic solvent, such as dimethylsulfoxide, dimethylformamide or dichloromethane, or mixtures thereof. Optionally, a solution or suspension of amine or diamine at a concentration of from 50 to 200 mg/mL may be used in step b). Therefore, the 2,9-(methylamide-substituted) minocycline and the amine or diamine may be reacted together when both in solution, when one is in solution and the other is in suspension or when both are in suspension. In addition, the 2,9-(methylamide-substituted) minocycline and the amine or diamine may be in solutions or suspensions comprising the same solvent or mixtures of solvents, or different solvents, or different combinations of solvents. Furthermore, the 2,9-(methylamide-substituted) minocycline and the amine or diamine may be in solutions or suspensions at the same or differing concentrations.

The 9-aminomethyl tetracycline intermediate used in step c) or d) may be in solution or suspension, optionally wherein the solution or suspension comprises a solvent selected from an alcohol, such as benzyl alcohol, ethanol or methanol, a polar aprotic solvent, such as dimethylsulfoxide, dimethylformamide or dichloromethane, or mixtures thereof. Optionally, a solution or suspension of 9-aminomethyl tetracycline intermediate at a concentration of from 20 to 100 mg/mL may be used in step c) or d). The aldehyde used in step c) may be in solution or suspension, optionally wherein the solution or suspension comprises a solvent selected from an alcohol, such as benzyl alcohol, ethanol or methanol, a polar aprotic solvent, such as dimethylsulfoxide, dimethylformamide or dichloromethane, or mixtures thereof. Optionally, a solution or suspension of aldehyde at a concentration of from 5 to 100 mg/mL may be used in step c). Therefore, the 9-aminomethyl tetracycline intermediate and the aldehyde may be reacted together when both in solution, when one is in solution and the other is in suspension or when both are in suspension. In addition, the 9-aminomethyl tetracycline intermediate and the aldehyde may be in solutions or suspensions comprising the same solvent or mixtures of solvents, or different solvents, or different combinations of solvents. Furthermore, the 9-aminomethyl tetracycline intermediate and the aldehyde may be in solutions or suspensions at the same or differing concentrations.

The concentration of the solutions or suspensions used in each of the steps of the method of the present invention will depend on the solubility of the reactants being used.

One advantage of running the method of the present invention as a continuous process in continuous flow reactors, such as pipe reactors, is that the volume of solvents are considerably reduced in comparison with those used in batch reactors. This in turn leads to a subsequent reduction in effluent, thus making these methods more environmentally friendly.

The use of continuous flow processes may provide the ability to perform chemical reactions with improved selectivity, reaction yields and product purity profile, reducing waste, being an environmentally friendly method to perform chemical synthesis. In many cases, continuous flow reactors may be utilized to further reduce the time and cost required to synthesize a desired product because they allow process intensification (e.g. high temperatures and pressure). For example, a continuous flow process may involve flowing a fluid sample comprising one or more precursor species into a flow-through system and performing a chemical reaction within the tubing of such a system to convert the precursor species into a desired product.

The use of a continuous flow reactor may provide the ability to use temperatures and pressures that are not readily attainable in batch processes. The use of elevated temperatures and pressures may facilitate conversion of the precursor species into a reaction product, without need for additives or promoter species.

In some cases, the reactions in steps a), b) and c) or d) are performed at a temperature of at least 10° C., at least 20° C., at least 75° C., at least 100° C., at least 125° C., at least 150° C., at least 175° C., at least 200° C., at least 225° C., at least 250° C., at least 275° C., at least 300° C., or, in some cases, greater. Optionally, the chemical reactions in steps a), b) and c) or d) are performed at a temperature of from 20° C. to 150° C., preferably from 20° C. to 120° C. Optionally, the reaction of step a) is performed at temperatures from 25° C. to 200° C., or from 25° C. to 50° C. Optionally, the reaction of step b) is performed at temperatures from 25° C. to 200° C., or from 100° C. to 200° C. Optionally, the reaction of step c) is performed at temperatures of at least 10° C., or from 20° C. to 80° C. Optionally, the reaction of step d) is performed at temperatures from 25° C. to 200° C., or from 20° C. to 50° C.

In some cases, the reactions in steps a), b) and c) or d) are performed at a pressure of at least 100 psi (689 KPa), at least 125 psi (862 KPa), at least 150 psi (1034175 KPa), at least 175 psi (1207 KPa), at least 200 psi (1379 KPa), at least 225 psi (1551 KPa), at least 250 psi (1724 KPa), at least 275 psi (1896 KPa), at least 300 psi (2068 KPa), at least 400 psi (2758 KPa), at least 500 psi (3447 KPa), or, in some cases, greater. Optionally, the reactions in steps a), b) and c) or d) are carried out at a pressure of from 100 to 2000 KPa. Optionally, the reactions in step b) is carried out at a pressure of at least 300 KPa, for example at a pressure of from 300 to 2000 KPa.

The term “flow through system” is used to refer to a system comprising one or more reactors which enable chemical reactions to occur in a continuous flow. The method of the present invention may be carried out in a pipe reactor, a plug flow reactor, a coil reactor, a tube reactor, a microchip, a continuous plate reactor, a packed bed reactor, a continuous stirred tank reactor (CSTR), or another commercially available continuous flow reactor, or a combination of two or more such reactors to form a flow through system. The flow through system may be designed and fabricated to be capable of withstanding a wide range of solvents and chemical conditions, including high temperature, high pressure, exposure to various solvents and reagents, and the like.

A continuous flow reactor can be made of any suitable compatible material comprising glass, different type of polymers (PFA, ETFE, PEEK, etc), Hastelloy®, silicon carbide, stainless steel and/or one or more high performance alloys. The continuous flow reactor may comprise static mixing apparatus. A continuous flow reactor may handle slurries, suitable for being subjected to temperature or temperature range and/or suitable for being subjected to pressure. Where one or more of the same continuous flow reactors or a combination of the different continuous flow reactors listed above are used, the reactors may be connected to one another such that fluid communication is possible. With respect to the term “connected”, this should be understood to mean that the continuous flow reactors need not necessarily be attached directly to one another, but the reactors should be in fluid communication with at least one other reactor. However, if desired, the reactors may be directly attached to each other.

One of the advantages of using a packed bed reactor, is that this type of reactor affords a higher effective molarity of any immobilized reagents, thereby decreasing reaction times. Moreover, any immobilized reagent is contained by the matrix, and consequently it is not necessary to separate the reaction mixture from such reagents In some cases, the reaction profile (e.g., reaction time, overall yield, distribution of reaction products, etc) may be substantially independent of fluid sample volume, such that the chemical reaction may be performed at larger scales without substantial change in reaction profile.

The method of the present invention may comprise at least one chemical reaction step carried out continuously with product isolation. Moreover, the method of the present invention may comprise at least two chemical reaction steps carried out in continuous-telescope mode with no reaction product/process intermediate isolation, only a change of solvent and/or removal of an excess of a reagent between the reaction steps. Further the product of the reaction step b) step is an intermediate that is used as a reactant in reaction step c) to prepare the desired product.

If desired, the method of the present invention may include one or more additional steps. The additional steps may comprise one or more washing steps, one or more purification steps, one or more isolation steps, or combinations thereof.

Where continuous flow reactors are used, the conditions within the one or more continuous flow reactors may be controlled. This may be done, for example, to enable a particular reaction to occur or to obtain a desired reaction rate. Controlling the conditions within the one or more continuous flow reactors may comprises adjusting or altering one or more of the following: the temperatures within the continuous flow reactor(s); the pressures within the continuous flow reactor(s); the solvents or solvent systems within the flow reactor(s); and flow rates within the continuous flow reactor(s). The concentration of each of the solutions or suspensions used in each of steps a), b) and c) or d) influences the flow rate, the residence time and the ratio of the reactants. Hence, adjusting or altering the concentration of each of the solutions or suspensions used in each of steps a), b) and c) or d) may also help control the conditions within the one or more continuous flow reactors.

The flow rate of reagents through a continuous flow reactor may be controlled, altered, or adjusted depending on the reaction carried out within the reactor. The flow rate of reagents may be different along one or more selected distances of a continuous flow reactor. The flow rate of reagents associated with a reaction step may affect the flow rate associated with the subsequent reaction step. Reagents may travel along a selected distance of a continuous flow reactor at different flow rates. The flow rates of reagents through the continuous flow reactor may be controlled, adjusted or altered using pumps.

As used herein, the term “reacting” refers to the forming of one or more bonds between two or more components to produce a stable, isolable compound (intermolecular reaction) or the forming of one or more bonds between two or more parts of the same molecule to form a stable, isolable compound (intramolecular reaction). That is, the term “reacting” does not refer to the interaction of solvents, catalysts, bases, ligands, or other materials, which may serve to promote the occurrence of the reaction with the component(s).

Each reaction is carried out in a heterogeneous or homogeneous environment. The continuous flow reactors may be adapted to carry out reactions in a heterogeneous and/or homogeneous environment. In particular, one or more continuous flow reactors may be adapted to carry out heterogeneous and/or homogeneous reactions. For example, the continuous flow reactors may comprise therein (e.g. within their bores) one or more reagents or catalysts. The catalysts may be homogenous or heterogeneous with respect to the reactants, reagents and/or solvents.

The reaction rate of each individual reaction step, the flow rate through each of the flow through systems and the rate of change of solvent can be adjusted so that flow through the whole system used to carry out the method of the present invention does not require the use of holding tanks at intermediates stages. Although, under certain circumstances, the use of holding tanks in downstream operations may be an option.

Where continuous flow reactors are used, the output of the one or more continuous flow reactors may be carefully controlled such that the composition with regards to the intermediate, reactants, impurities and solvents etc. is suitable to be fed into a subsequent continuous flow reactors to allow for optimal reaction conditions.

Where a flow through system is used, the conditions in the flow through system (e.g. a systems comprising multiple tubular reactors) may vary over a wide range. In particular, the conditions may vary from homogeneous reaction conditions to heterogeneous conditions. For example, a heterogeneous reaction may be used in the reductive amination reaction in step c) where the continuous flow reactor, such as a tubular reactor, is filled with a heterogeneous catalyst. The heterogeneous catalyst may, for example be a reducing agent.

Also, continuous solvent extraction/wash steps or membrane purification may be applied to remove impurities, excess reagents, or other undesirable materials, which could be detrimental to subsequent chemical reactions or to the purity of the final product. The pressure in each of the reactors within a flow through system may be atmospheric or above atmospheric pressure and temperatures can vary from below ambient to above 200° C.

Purification, isolation and drying of the final product, when required, can also be carried out in a continuous fashion using continuous extraction, membranes, crystallization, filtration, and drying processes.

### EXAMPLES

### Example 1

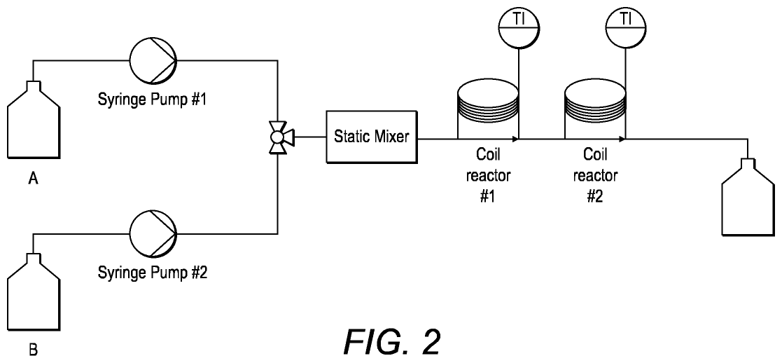

Flow experiments were performed using the continuous flow setup shown in FIG. 2 (in which “TI” means temperature instrument, Coil Reactor #1 is the reaction coil and Coil Reactor #2 is the cooling coil). Solution A was prepared by dissolving minocycline (10.00 g) in sulfuric acid (50 mL). Solution B was prepared by dissolving N′-(hydroxymethyl) phthalimide (7.75 g) in sulfuric acid (50 mL). Solutions A and B were pumped in a 2:1 ratio to achieve 5 minutes residence in a coil reactor (504 μL) at 80° C. The resulting solution showed a conversion of 100% of the starting material resulting in 50% of 2,9-methylphtalimide minocycline and 50% of 2-methylphtalimide minocycline.

### Example 2

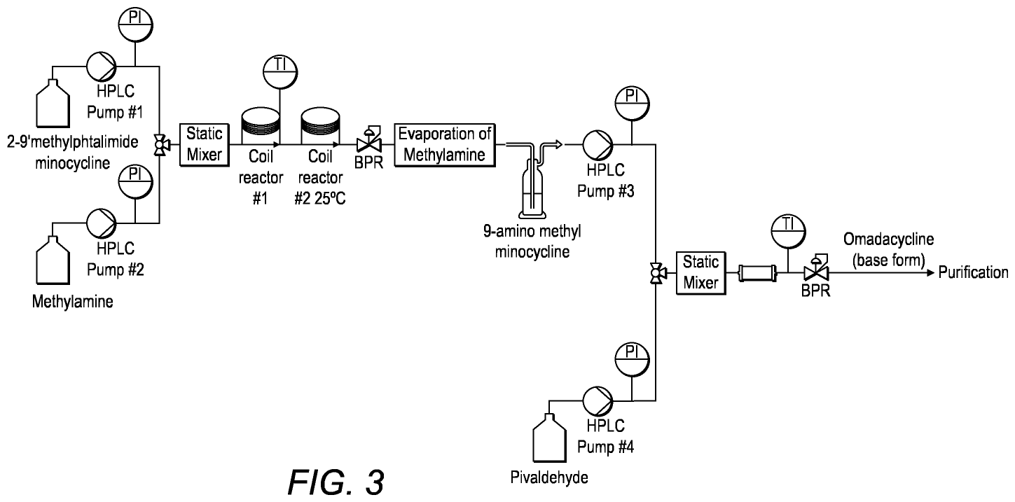

Flow experiments were performed using the continuous flow setup shown in Error! Reference source not found. FIG. 3 (in which “TI” means temperature instrument, “PI” means pressure instrument, “BPR” means back pressure regulator, “HPLC pump” means high performance liquid chromatography pump, Coil Reactor #1 is the reaction coil and Coil Reactor #2 is the cooling coil). Solution A was prepared by dissolving 2,9-methylphtalimide minocycline (15.00 g) in benzyl alcohol (150 mL). Solution B was prepared by mixing methylamine ethanolic solution (33%) (41.85 g) and benzyl alcohol (94.65 mL). Solutions A and B were pumped in a 1:1 ratio to achieve 6 minutes residence time in a coil reactor (10 mL) at 115° C. and 7 bar (700 KPa) of back-pressure. The product stream was collected at the outlet in a round bottom flask containing absolute ethanol (200 mL) at 30° C. Distillation of the mixture provided a solution with a residual amount of methylamine (solution C). Solution D was prepared by mixing pivaldehyde (4.90 mL), triethylamine (2.52 mL) and benzyl alcohol (292.6 mL). Solutions C and D were pumped in a 1:1 ratio and thoroughly mixed to achieve 30 minutes residence time in a packed bed reactor containing immobilized sodium cyanoborohydride (25 g) at a temperature of 25° C. Once the steady state was achieved, fractions were collected and diluted properly for HPLC analysis. The resulting solution showed a conversion of 78%.

### Example 3

Flow experiments were performed using the continuous flow setup shown in FIG. 3. Solution A was prepared by dissolving (4S,12aS)-9-(aminomethyl)-4,7-bis(dimethylamino)-3,10,12,12a-tetrahydroxy-1,11-dioxo-4a,5,5a,6-tetrahydro-4H-tetracene-2-carboxamide (0.50 g, 1 mmol). in dichloromethane (3 mL). Solution B was prepared by mixing triethylamine (0.21 g, 2.1 mmol) and 1-chloro-2,2-dimethyl-propane (0.22 g, 2.1 mmol) in dichloromethane (3 mL). Solutions A and B were pumped in a 1:1 ratio to achieve 1 hour residence time in a coil reactor (1 mL) at 35° C. and 2 bar of back-pressure. A quantitative yield of raw material was produced.

### Example 4

Lab-scale nanofiltration equipment (MetCell Cross Flow System) was used for the separation of methylamine, reaction by-products and the 9-aminomethyl tetracycline intermediate. The membrane disk was prepared according to the filtration cell diameter and the system was assembled with compatible o-rings. A crude solution of 9-aminomethyl tetracycline intermediate was added to the METCell tank base and recirculated for 10 minutes. 30 bar (3000 KPa) pressure was applied to the system and the permeate flow rate was calculated with a chronometer (Qperm=0.133 mL/min). A new solution of ethanol was fed into the system over 5 hours with a constant flow rate of 0.2 mL/min. The permeate samples and the final retentate solution were analyzed by HPLC and GC. 200 mL retentate solution was obtained (45 wt % BnOH, 5 wt % methylamine, 43% EtOH, 8 wt % solutes). The membrane rejection for the 9-aminomethyl tetracycline intermediate was 99% during this operation step.

### Example 5

Feed 1 contained a benzyl alcohol solution of 9-aminomethyl tetracycline intermediate (3.3 mmol, 55 mL), and feed 2 contained pivaldehyde (1.08 mL), triethylamine (0.46 mL), and benzyl alcohol to reach 55 mL. Both streams were pumped using two high-pressure liquid pumps (P1 and P2, Knauer) at 0.1 mL/min each. The two liquid streams were combined in a T-mixer and mixed in a Uniqsis glass static mixer (578 μL, T: 175 s): before entering a packed bed reactor (T: 30 minutes) containing immobilized sodium cyanoborohydride (5.33 g, 84.8 mmol). The packed bed was placed on an HPLC oven heated to 25° C. The HPLC pump flow rate and pressure were measured and monitored by the control platform of the pumping system. Once the steady-state was achieved, fractions were collected and diluted properly for HPLC analysis. Conversion and yield were determined by HPLC. The combined fractions measured 75.53% conversion.

### Example 6

The reaction solution was added slowly to a flask containing a mixture of methyl tert-butyl ether (MTBE) and n-heptane. The solid was filtered, washed with MTBE, and dried on a stove until constant weight was reached with a temperature not higher than 30° C. and nitrogen sweep. The solid was re-suspended in i-PrOH (70 mL, 7 mL/g), and p-toluenesulfonic acid was added to the suspension that was then stirred for 24 hours at 550 rpm and at room temperature, under a nitrogen atmosphere. Omadacycline tosylate was obtained in 77.08% mol yield.

What is claimed is:

1-33. (canceled)

34. A method for synthesizing 9-aminomethyl tetracycline compounds according to Formula 3, wherein R is a hydrogen or a C1 to C10 straight chain alkyl group, a C3-C20 branched chain alkyl group, a substituted C1 to C10 straight chain alkyl group, a substituted C3 to C20 branched alkyl group, a C3 to C10 aryl group, a substituted C3 to C10 aryl group, or a C3 to C10 heteroaryl group comprising at least one oxygen, nitrogen, sulfur or phosphorous atom 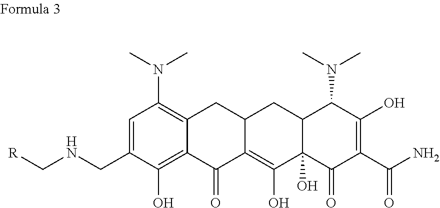
the method comprising:
a) reacting minocycline and an hydroxymethylamide derivative at a temperature of from 20° C. to 120° C. to form a 2,9-(methylamide-substituted) minocycline and a 2-(methylamide-substituted) minocycline;
b) reacting the 2,9-(methylamide-substituted) minocycline from step a) and an amine at a temperature of from 100° C. to 200° C. to form a 9-aminomethyl tetracycline intermediate;
wherein the amine is in accordance with Formula 5 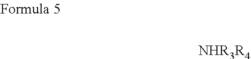
wherein R₃ and R₄ is a hydrogen atom, a C1-C10 straight chain alkyl group, a C3-C20 branched chain alkyl group, or a substituted C1 to C10 alkyl group, a substituted C3 to C20 branched alkyl group; and
c) reacting the 9-aminomethyl tetracycline intermediate from step b) and an aldehyde in the presence of a reducing agent at a temperature of from 20° C. to 80° C. to form a 9-aminomethyl tetracycline compound; or
d) reacting the 9-aminomethyl tetracycline intermediate from step b) and an alkyl halide or an alkylating reagent at a temperature of from 20° C. to 50° C. to form a 9-aminomethyl tetracycline compound;
wherein the method is a semi continuous or continuous flow process.

35. The method of claim 34, wherein R is a C6 to C10 aryl group or a substituted C6 to C10 aryl group.

36. The method of claim 34, wherein step b) is operated in the absence of a hydrogenation reaction.

37. The method of claim 34, wherein (i) steps a) and b) of the method of the present invention operate in a continuous manner, (ii) steps b) and c) of the method of the present invention operate in a continuous manner, or (iii) steps b) and d) of the method of the present invention operate in a continuous manner.

38. The method of claim 34, wherein the residence time of the reactions in steps a), b) and c) or d) is from 12 seconds to 30 minutes.

39. The method of claim 34, wherein the reactions in steps a), b) and c) or d) are carried out in a pipe reactor, a plug flow reactor, a coil reactor, a tube reactor, a microchip, a continuous plate reactor, a packed bed reactor, a continuous stirred tank reactor (CSTR), or another commercially available continuous flow reactor, or a combination of two or more such reactors.

40. The method of claim 34, wherein the minocycline in step a) is in solution or suspension, optionally wherein the solution or suspension comprises a solvent selected from an organic acid or mineral acid such as sulfuric acid, methanesulfonic acid, triflic acid, sulfuric acid fuming 65% SO₃ or mixtures thereof.

41. The method of claim 34, wherein the hydroxymethylamide derivative in step a) is in solution or suspension, optionally wherein the solution or suspension comprises a solvent selected from an organic acid or mineral acid such as sulfuric acid, methanesulfonic acid, triflic acid, sulfuric acid fuming 65% SO₃.

42. The method of claim 34, wherein the hydroxymethylamide derivative in step a) is in accordance with Formula 4 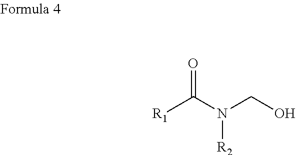
wherein R, is a C1-C10 straight chain alkyl group, a C3-C20 branched chain alkyl group, a C2-C10 straight chain alkenyl group, a C3-C20 branched chain alkenyl group, a C2-C10 straight chain alkynyl group, a C3-C20 branched chain alkynyl group, a C3 to C10 aryl group, a C3 to C10 heteroaryl group comprising at least one oxygen, nitrogen, sulfur or phosphorous atom, or an halogen selected from chlorine, bromine and iodine; and R₂ is a C1-C10 straight chain alkyl group, a C3-C20 branched chain alkyl group, a C2-C10 straight chain alkenyl group, a C3-C20 branched chain alkenyl group, a C2-C10 straight chain alkynyl group, a C3-C20 branched chain alkynyl group, a C3 to C10 aryl group, or a C3 to C10 heteroaryl group comprising at least one oxygen, nitrogen, sulfur or phosphorous atom, optionally wherein R₂ is linked to R₁ to form a 4-8 membered ring, and optionally wherein the ring is substituted and comprises carbon atoms and/or heteroatoms such as oxygen, nitrogen, and sulfur.

43. The method of claim 34, wherein the hydroxymethylamide derivative in step a) is N′-hydroxymethyl-phthalimide.

44. The method of claim 34, wherein the 2,9-(methylamide-substituted) minocycline in step b) is in solution or suspension, optionally wherein the solution or suspension comprises a solvent selected from an alcohol, such as benzyl alcohol, a polar aprotic solvent, such as dimethylsulfoxide, dimethylformamide or dichloromethane, or mixtures thereof.

45. The method of claim 34, wherein the amine in step b) is in solution or suspension, optionally wherein the solution or suspension comprises a solvent selected from an alcohol, such as benzyl alcohol, a polar aprotic solvent, such as dimethylsulfoxide, dimethylformamide or dichloromethane, or mixtures thereof.

46. The method of claim 34, wherein R₃ and R₄ of the amine are selected from a C1-C4 straight chain alkyl group, a C3-C4 branched chain alkyl group, or a substituted C1-C4 straight chain alkyl group or a substituted C3-C4 branched chain alkyl group.

47. The method of claim 34, wherein the amine in step b) is selected from methylamine, ethanolamine and n-propylamine.

48. The method of claim 34, wherein an excess of amine is used in step b).

49. The method of claim 48, wherein the excess of amine is continuously removed prior to step c) or d).

50. The method of claim 34, wherein the 9-aminomethyl tetracycline intermediate in step c) or d) is in solution or suspension, optionally wherein the solution or suspension comprises a solvent selected from an alcohol, such as benzyl alcohol, ethanol or methanol, a polar aprotic solvent, such as dimethylsulfoxide, dimethylformamide or dichloromethane, or mixtures thereof.

51. The method of claim 34, wherein the aldehyde in step c) is in solution or suspension optionally wherein the solution or suspension comprises a solvent selected from an alcohol, such as benzyl alcohol, ethanol or methanol, a polar aprotic solvent, such as dimethylsulfoxide, dimethylformamide or dichloromethane, or mixtures thereof.

52. The method of claim 34, wherein the aldehyde in step c) is in accordance with Formula 6 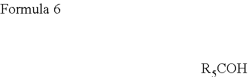
wherein R₅ is a hydrogen, a C1-C10 straight chain alkyl group, a C3-C20 branched chain alkyl group, a substituted C1 to C10 straight chain alkyl group, a substituted C3 to C20 branched alkyl group, a C3 to C10 aryl group, a substituted C3 to C10 aryl group or a C3 to C10 heteroaryl group comprising at least one oxygen, nitrogen, sulfur or phosphorous atom.

53. The method of claim 34, wherein the aldehyde in step c) is selected from pivaldehyde, acetaldehyde and benzaldehyde.

54. The method of claim 34, wherein the reducing agent in step c) is an immobilized reducing agent.

55. The method of claim 54, wherein the immobilized reducing agent is immobilized sodium cyanoborohydride.

56. The method of claim 34, wherein the alkyl halide is in accordance with Formula 7 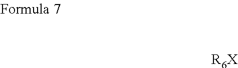
wherein R₅ can be a C1-C10 straight chain alkyl group, a C3-C20 branched chain alkyl group, a substituted C1 to C10 straight chain alkyl group, a substituted C3 to C20 branched alkyl group, a C3 to C10 aryl group, a substituted C3 to C10 aryl group or a C3 to C10 heteroaryl group; comprising at least one of oxygen, nitrogen, sulfur or phosphorous atom and X is an halogen selected from chlorine, bromine and iodine.

57. The method of claim 56, wherein the alkyl halide is selected from 1-chloro-2,2-dimethylpropane, 1-bromo-2,2-dimethylpropane and 1-iodo-2,2-dimethylpropane.

58. The method of claim 34, wherein reaction step c) or d) is carried out in the presence of a proton acceptor.

59. The method of claim 58, wherein the proton acceptor is selected from triethylamine, ammonia and 4-dimethylaminopyridine.

60. The method of claim 34, wherein reaction step c) or d) is carried out in the presence of an organic acid, such as formic acid or acetic acid, an inorganic acid or mixtures thereof.

61. The method of claim 34, wherein the reactions in steps a), b), c) and/or d) are carried out at a pressure of from 100 to 2000 KPa.

62. The method of claim 34, wherein the 9-aminomethyl tetracycline compound formed in step c) or d) is omadacycline.

63. The method of claim 34, wherein, following step c) or d), counter ion exchange is performed to form an omadacycline salt.

64. The method of claim 62, wherein the omadacycline formed has a purity higher than 50%, optionally between 70 and 80% or between 81 and 100%.

65. The method of claim 63, wherein the omadacyline salt formed has a purity higher than 50%, optionally between 70 and 80% or between 81 and 100%.

66. The method of claim 64, wherein the omadacycline formed has an epimer content of less than 10%, optionally less than 2%.

67. The method of claim 65, wherein the omadacyline salt formed has an epimer content of less than 10%, optionally less than 2%.
